(19) United States
(12) Patent Application Publication
(10) Pub. No.: US 2018/0271976 A1
(43) Pub. Date: Sep. 27, 2018

(54) METHODS FOR GENERATING NEUTRALIZING ANTI-PATHOGEN ANTIBODIES BY DIRECTING THE HUMORAL RESPONSE TOWARD DESIRED EPITOPES

(71) Applicant: NORTHEASTERN UNIVERSITY, Boston, MA (US)

(72) Inventors: Murillo Lobo Gomes SILVA, Marlborough, MA (US); Michail V. SITKOVSKY, Boston, MA (US)

(21) Appl. No.: 15/919,866

(22) Filed: Mar. 13, 2018

Related U.S. Application Data
(60) Provisional application No. 62/470,868, filed on Mar. 14, 2017.
(60) Provisional application No. 62/473,270, filed on Mar. 17, 2017.

(51) Int. Cl. A61K 39/385; A61K 39/12; A61K 39/145; A61K 39/015; A61K 47/64

(52) U.S. Cl. CPC A61K 39/385; A61K 39/12; A61K 2039/545; A61K 39/015; A61K 47/646; A61K 39/145

(57) ABSTRACT

Methods for generating antibodies to immuno-subdominant epitopes of elusive pathogens and vaccines and methods of vaccinating subjects against elusive pathogens are disclosed. The methods include targeting immunodominant B cells to undergo apoptosis, for example, while simultaneously targeting the germinal center (GC) reaction toward preferential selection and differentiation of B cells that produce broadly neutralizing antibody against a targeted pathogen.

### CROSS REFERENCE TO RELATED APPLICATIONS

This application is entitled to priority pursuant to 35 U.S.C. § 119(e) to U.S. provisional patent application No. 62/470,868, filed Mar. 14, 2017 and U.S. provisional patent application No. 62/473,270, filed Mar. 17, 2017. The entire disclosure of each of the afore-mentioned patent applications is incorporated herein by reference.

### STATEMENT REGARDING FEDERALLY SPONSORED RESEARCH OR DEVELOPMENT

This invention was made with government support under U19 AI 091693 awarded by the National Institutes of Health. The government has certain rights in the invention.

### FIELD OF THE INVENTION

The invention relates to vaccines and methods for immunizing subjects against elusive pathogens. The invention also relates to methods of generating neutralizing antibodies to immuno-subdominant epitopes of elusive pathogens. The invention also provides methods of producing therapeutic antibodies that bind to specific epitopes of therapeutic targets.

### BACKGROUND OF THE INVENTION

The majority of approved vaccines function through the induction of long-lived neutralizing antibody (Ab) responses (Hangartner et al., 2006; Plotkin, 2010) in which the Ab binds to neutralizing sites (epitopes) of a pathogen. Pathogens that can readily mutate, such as influenza virus or human immunodeficiency virus (HIV), can evade the immune response elicited by the vaccine. And as a result, conventional vaccines have not been very effective for such elusive pathogens. Specific sites within proteins of pathogens such as Influenza virus or HIV that are required for viral function have been identified, and these sites or epitopes are highly conserved between the divergent strains of each particular virus. Researchers have also identified a subset of patients with chronic HIV infection have developed broadly-neutralizing antibodies (bnAb) that specifically bind to these highly conserved regions of HIV. These antibodies have been shown to prevent infection from over 70% of known HIV strains and are highly protective. Developing a vaccination strategy or vaccine that is capable of inducing a similar response is highly desired, but after decades of research, these endeavors have not been successful.

A long-lasting and effective Ab response is derived from the germinal center (GC). The micro-anatomical structure of GCs is critical to the development of high affinity anti-pathogen antibodies. B cells of differing antigen specificities and affinities compete for the limited amount of growth resources available in the GCs. B cell survival and propagation are based on B cell receptor affinity toward a particular antigen. Those cells that bind to antigen very effectively are selected to expand. Somatic hypermutation of immunoglobulin genes along with iterative cycles of clonal selection, drive an increase in Ab affinity over the course of an immune response. GC B cells differentiate into long-lived Ab-secreting plasma cells and memory B cells; both critical cell types to an effective immune/vaccine response.

A T cell-based selection mechanism is, at least in part, responsible for regulating initial B cell entry and selection in the GC (Schweikert et al., 2011; Victora et al., 2010). This selection process predominantly favors the entry of high-affinity clones, which are able to capture large amounts of antigen and display high densities of peptide-MHC II to a limited number of cognate T follicular helper s (Tfh) cells. Although this competitive selection process is required for affinity maturation, it likely limits the diversity of B cell clones that can participate in the GC reaction (DalPorto et al., 2002) and skews the immune response toward immunodominant epitopes (Havenar-Daughton, 2016).

Immunodominance appears to be an important factor in the failure to generate long-term protective immunity to elusive pathogens (Ellebedy et al., 2014; Haynes et al., 2012). Adjuvants that promote strong Tfh responses, such as MF59, are in part capable of overcoming immunodominance and have been demonstrated to increase the breadth of the immune response (Zedda et al., 2015). However, since the immune system does not distinguish between the biological significance of non-neutralizing, narrowly neutralizing and broadly neutralizing B cell specificities, a rational strategy to coerce the response to overcome immunodominance while simultaneously favoring the expansion of specific bnAb precursor clones must be implemented (Haynes et al., 2012).

There is need in the art for novel approaches for the generation of improved vaccines. Specifically, there is a need for methods to generate antibodies to specific or desired epitopes of a particular antigen of a pathogen. Useful epitopes, i.e., neutralizing epitopes, for vaccine development may be more highly conserved than immunodominant (non-neutralizing) epitopes. Methods for generating antibodies to neutralizing epitopes may be applied to the generation of antibodies for a wide range of antigens and protein targets, or vaccines for numerous human diseases, such as infections caused by viruses and microorganisms, as well as cancer.

Current strategies for developing therapeutic monoclonal antibodies require extensive screening to isolate B cells that produce antibodies with desired functionalities. The rational epitope targeting strategy described herein significantly reduces screening time and provides a method for producing antibodies against epitopes which would otherwise not develop naturally (i.e. immune-subdominant epitopes).

### SUMMARY OF THE INVENTION

In one aspect of the invention, there is provided a method of immunizing a mammalian subject against a pathogen. The method comprises the steps of
(1) administering a primary immunization composition to the subject, said primary immunization composition comprising a peptide comprising a peptide expressed by the pathogen, wherein said peptide comprises a neutralizing epitope; and
(2) administering to the subject a composition comprising an inhibitory construct comprising at least one copy of an altered form of the peptide, wherein the altered form comprises at least one amino acid change or structural alteration of the neutralizing epitope that prevents binding of neutralizing antibody thereto; and
   wherein the inhibitory construct is administered and prior to, concurrently with or within two months of administration of the primary immunization composition. In certain embodiments of this aspect of the invention, the pathogen is selected from HIV, influenza virus, shigella, herpes virus, plasmodium species, dengue virus, mycobacterium tuberculosis, cytomegalovirus, respiratory syncytial virus, ebola virus, hepatitis C, West Nile virus, streptococcus pneumoniae, streptococcus aureus, and streptococcus pyrogenes.

In another aspect of the invention, there is provided a method of generating therapeutic antibodies to a specific epitope of a therapeutic target, comprising the steps of:
(1) administering a primary immunization composition to a mammal, said primary immunization composition comprising a recombinant peptide comprising the epitope of the therapeutic target;
(2) administering to the subject a composition comprising an inhibitory construct comprising at least one copy of an altered form of the recombinant peptide, wherein the altered form comprises at least one amino acid change or structural alteration of the epitope that prevents binding of antibody thereto; and wherein the inhibitory construct is administered separately from and prior to, concurrently with or within two months of administration of the primary immunization composition;
(3) Isolating one or more B cells from said mammal;
   wherein an antibody expressed by the one or more B cells comprises a variable domain and said antibody binds to the epitope; and
(4) Identifying the nucleic acid sequence that encodes the variable domain of said antibody and utilizing said sequence to generate a recombinant version of the antibody.
   In certain embodiments of this aspect, the therapeutic agent is selected from the group consisting of a cytokine, an enzyme, a chemokine, a hormone, a receptor, and a receptor ligand.

In yet another aspect of the invention, there is provided a vaccine for a mammalian pathogen. The vaccine comprises (1) a first composition comprising a peptide comprising at least one copy of a primary immunization composition, said primary immunization composition comprising a recombinant peptide comprising a peptide expressed by the pathogen, wherein said peptide comprises a neutralizing epitope; and (2) a second composition comprising an inhibitory construct comprising at least one copy of an altered form of the peptide, wherein the altered form comprises at least one amino acid change or structural alteration of the neutralizing epitope that prevents binding of neutralizing antibody thereto. In certain embodiments, the vaccine is for a pathogen such as HIV, influenza virus, shigella, herpes virus, plasmodium species, dengue virus, mycobacterium tuberculosis, cytomegalovirus, respiratory syncytial virus, ebola virus, hepatitis C, West Nile virus, streptococcus pneumoniae, streptococcus aureus, and streptococcus pyrogenes.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### Definitions

The articles “a” and “an” are used herein to refer to one or to more than one (e.g., to at least one) of the grammatical object of the article. By way of example, “an element” means one element or more than one element.

The term “about” will be understood by persons of ordinary skill in the art and will vary to some extent depending on the context in which it is used. As used herein, “about” is meant to encompass variations of plus or minus 10%, more preferably plus or minus 5%, even more preferably 1%, and still more preferably plus or minus 0.1%.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies that may be used in the practice of the present invention may exist in a variety of forms including, for example, polyclonal antibodies, monoclonal antibodies, Fv, Fab and F(ab)2, as well as single chain antibodies and humanized antibodies (Harlow et al., 1999, Using Antibodies: A Laboratory Manual, Cold Spring Harbor Laboratory Press, New York; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The expression “amino acid” as used herein is meant to include both natural and synthetic amino acids, and both D and L amino acids.

The term “immunization” refers to a process to induce partial or complete protection against a disease. Alternatively, the term refers to a process to induce or amplify an immune system response to an antigen.

An “immune response” to an antigen or vaccine composition is the development in a subject of a response including a humoral and/or a cell-mediated immune response to molecules present in an antigen or a vaccine composition of interest. For purposes of the present invention, a “humoral immune response” is an antibody-mediated immune response and involves the generation of antibodies with affinity for the antigen/vaccine of the invention, while a “cell-mediated immune response” is one mediated by T-lymphocytes and/or other white blood cells.

A “mutation,” as used herein, refers to a change in nucleic acid or polypeptide sequence relative to a reference sequence (which is preferably a naturally-occurring normal or “wild-type” sequence), and includes translocations, deletions, insertions, and substitutions/point mutations. A “mutant,” as used herein, refers to either a nucleic acid or protein comprising a mutation.

As used herein, the terms “peptide,” “polypeptide,” and “protein” are used interchangeably, and refer to a compound comprised of amino acid residues covalently linked by peptide bonds. A protein or peptide contains two or more amino acids, while no limitation is placed on the maximum number of amino acids which can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptide, homodimers, heterodimers, variants of polypeptides, modified polypeptides, derivatives, analogs, fusion proteins, among others. The polypeptides include natural peptides, recombinant peptides, synthetic peptides, or a combination thereof.

“Recombinant protein” means a protein that results from the expression of recombinant DNA within living cells.

“Pharmaceutically acceptable” means physiologically tolerable, for either human or veterinary applications.

As used herein, “pharmaceutical compositions” include formulations for human and veterinary use.

The term “vaccine” as used herein is defined as a material used to provoke an immune response after administration of the material to a subject, typically a mammal. The vaccine can include one or more immunogenic compositions, e.g., a primary immunogenic composition together with a secondary immunogenic composition, e.g., a composition containing an inhibitory construct. Immune response includes responses that result in at least some level of immunity in the subject to which the immunogenic composition is administered.

The term “inhibitory construct” as used herein is defined as a primary immunogenic recombinant peptide further comprising at least one copy of a mutation or alteration in structure which prevents antibody binding to the targeted epitope.

As used herein, the term “epitope” is meant to refer to any antigenic determinant on an immunogen, e.g., any primary immunogen, to which an antibody binds through an antigenic binding site. Determinants or antigenic determinants on an antigen usually consist of chemically active surface groupings of molecules such as amino acids or sugar side chains and usually have specific three-dimensional structural characteristics, as well as specific charge characteristics. In some cases, an epitope may be an area of surface exposed residues and/or carbohydrate moieties on an antigen.

As used herein, “neutralizing epitope” means the region of a protein (antigen) naturally expressed by a pathogen where the binding of an antibody to this region can effectively constrain pathogen infectivity.

“Immunodominance” is the immunological phenomenon in which immune responses are mounted against only a few of the antigenic peptides out of the many produced. An “immunodominant” antigenic determinant is an epitope that is most easily recognized by the immune system and thus most influences the specificity of the induced antibody. Immunodominant epitopes are generally non-neutralizing.

### BRIEF DESCRIPTION OF THE DRAWINGS

FIGS. 1A-B are schematics showing the immunization strategy described herein. FIG. 1A shows the response obtained with the present immunization strategy (HIV is used as an example) where the immune response is dominated by B cells that bind to non-neutralizing epitopes. FIG. 1B represents the strategy to specifically inhibit unwanted B cell propagation to ensure that neutralizing B cells obtain a survival advantage in the competitive germinal center environment.

FIGS. 2A-B show alternative strategies for inhibiting non-neutralizing antibodies. FIG. 2A: depicts a liposomal formulation where the mutated recombinant protein used for B cell inhibition (the inhibitory construct) is displayed on the liposome surface and the inhibitor signaling molecule is encapsulated and/or incorporated on the liposomal bilayer. FIG. 2B depicts an inhibitor molecule directly conjugated to the mutated inhibitory construct.

FIGS. 3A-D show that germinal center competition drives the B cell response toward immunodominant epitopes. C57bl/6j mice were immunized i.p. with 10 μg of either NP₄-OVA or OVA in precipitated alum. Spleens were dissected 12 days after immunization. FIG. 3A: Gating strategy for antigen specific germinal center B cells (B220⁺, CD4⁻, CD38^{lo}, GL7⁺, OVA⁺/NP⁻ or OVA⁻/NP⁺) and representative plots from each group. FIG. 3B, FIG. 3C: Quantification of antigen specific B cell frequency and cell count. FIG. 3D: Germinal center B cell frequency after each immunization. Cell counts are normalized to 10⁶ lymphocytes. Bars represent mean; NS, not significant; **p<0.01, ***p<0.001, unpaired Welch's t test.

FIGS. 4A-H show that soluble antigen treatment reduces the number of immunodominant B cells in germinal centers and favors the expansion of subdominant cells. FIG. 4A: Schematic outline of experimental approach for FIG. 4 A-F where mice were immunized i.p. with 10 μg of NP₄-OVA in precipitated alum then treated with soluble NP-Ficoll or PBS on days 6 through 8. Spleens were dissected either 9, 12, or 21 days after immunization. FIGS. 4B-C: Representative plots of GC frequency (B220⁺, CD4⁻, GL7⁺, CD38^{lo}) and quantification. FIG. 4D: Representative gating for antigen specific germinal center B cells (B220⁺, GL7⁺, CD4″, CD38^{lo}, and OVA⁺/NP⁻ or OVA⁻/NP⁺). FIGS. 4E, 4F: Quantification of NP-specific (E) and OVA-specific (F) germinal center B cell frequency and cell number. FIGS. 4G, 4H: Mice were immunized i.p. with 1 μg of CRM-OVA then treated with soluble OVA or PBS on day 7. Spleens were dissected 12 days after immunization. (G) Representative plots of antigen specific GC B cells and quantification of CRM⁺ cells (H). Cell counts are normalized to 10⁶ lymphocytes. Bars represent mean; NS, not significant; *p<0.05, **p<0.01, ***p<0.001, Welch's t test

FIGS. 5A-H show that NP-Ficoll treatment shifts OVA⁺ GC B cell population to dark zone (DZ).

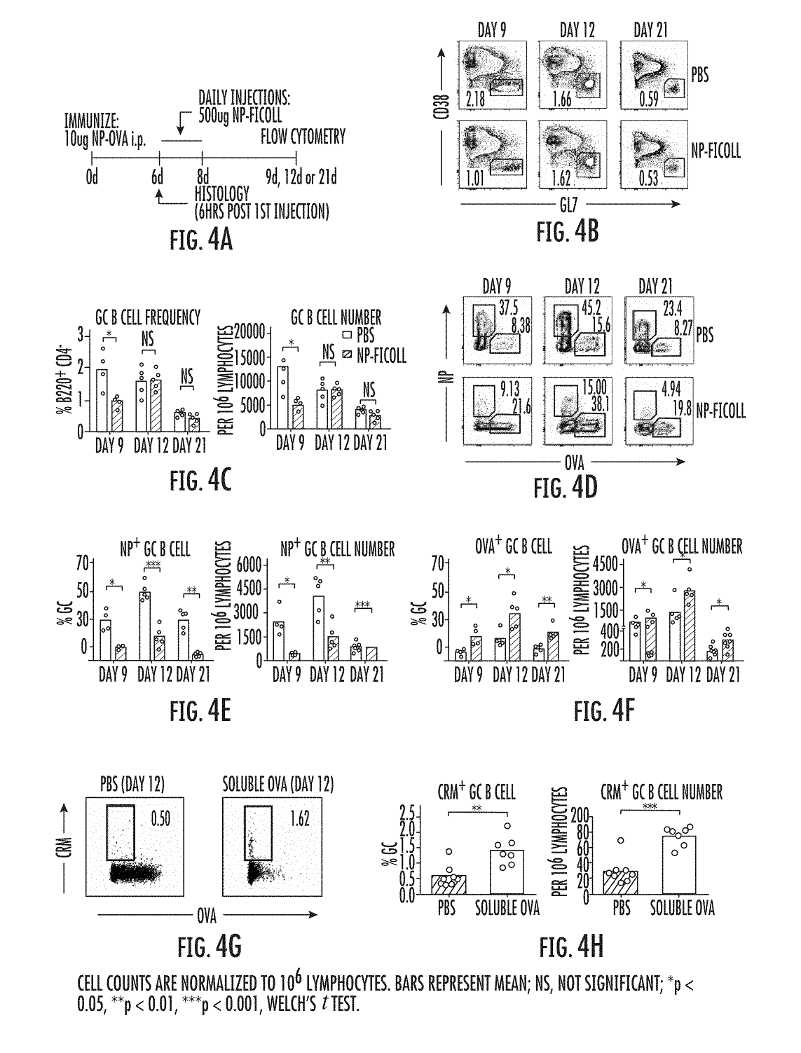

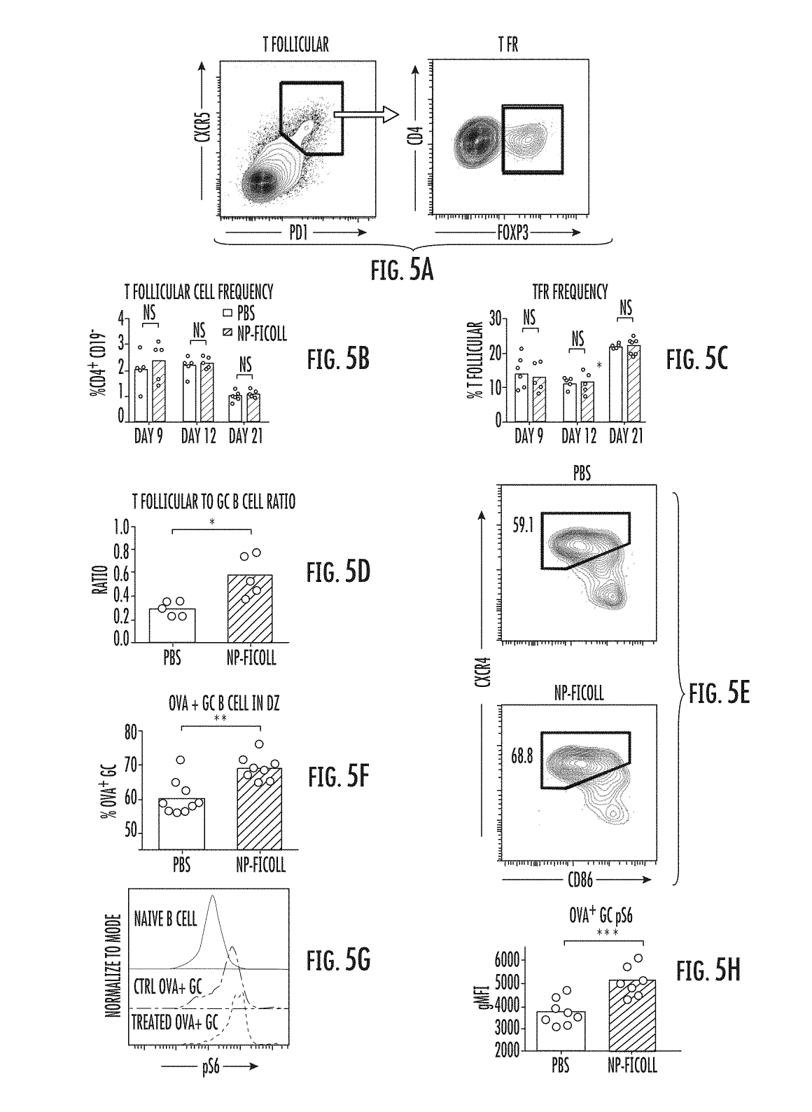

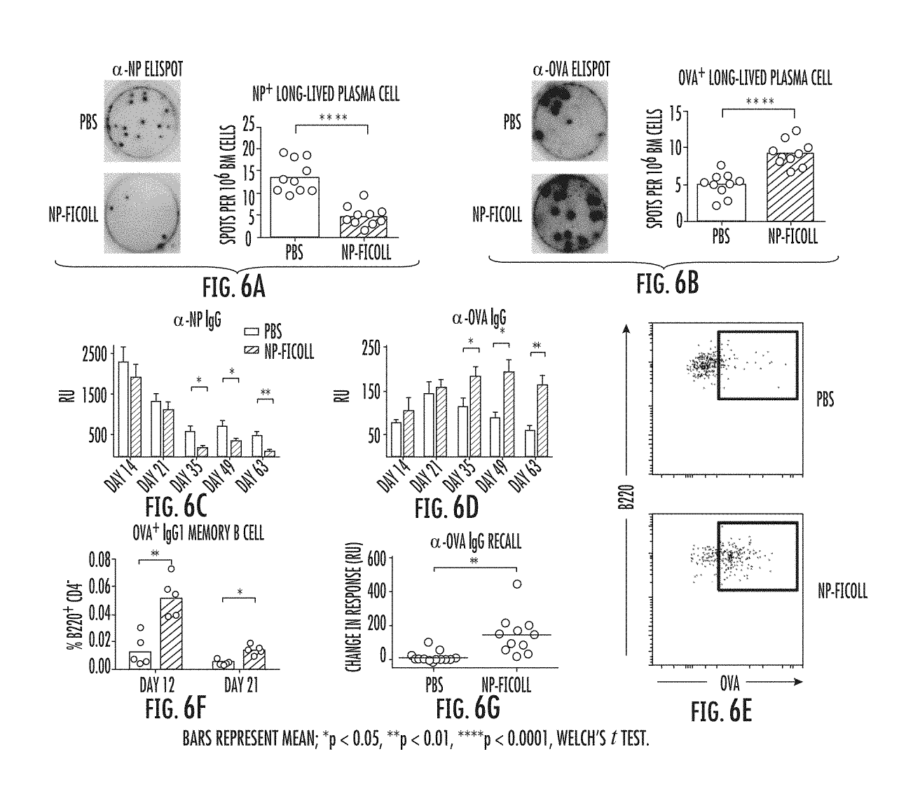

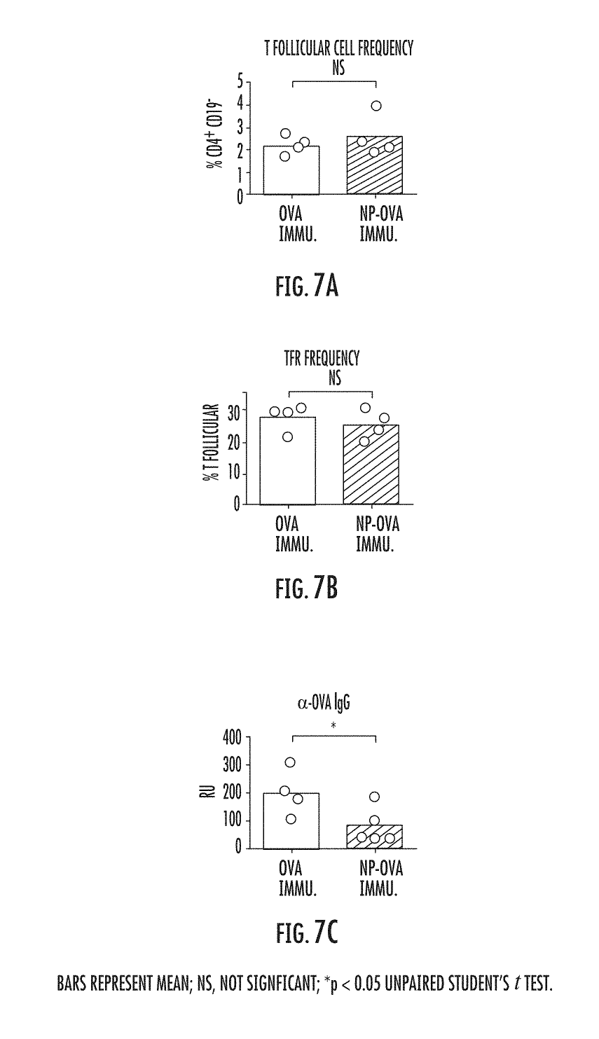

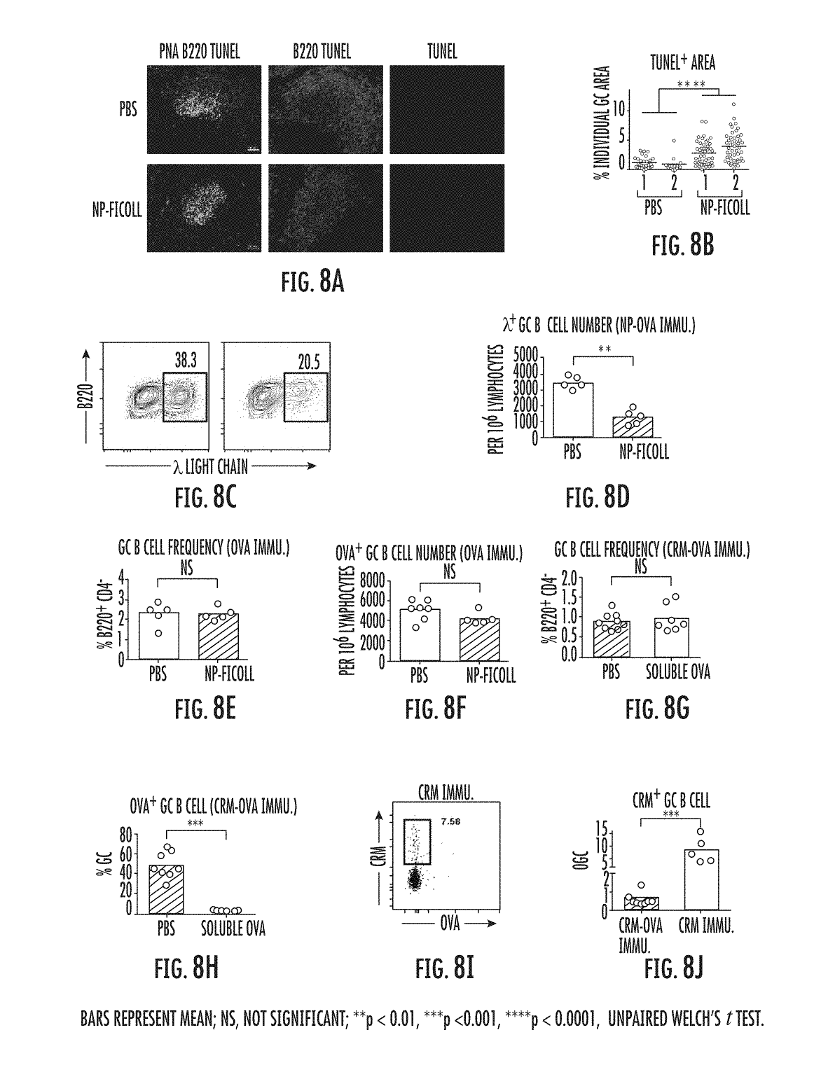

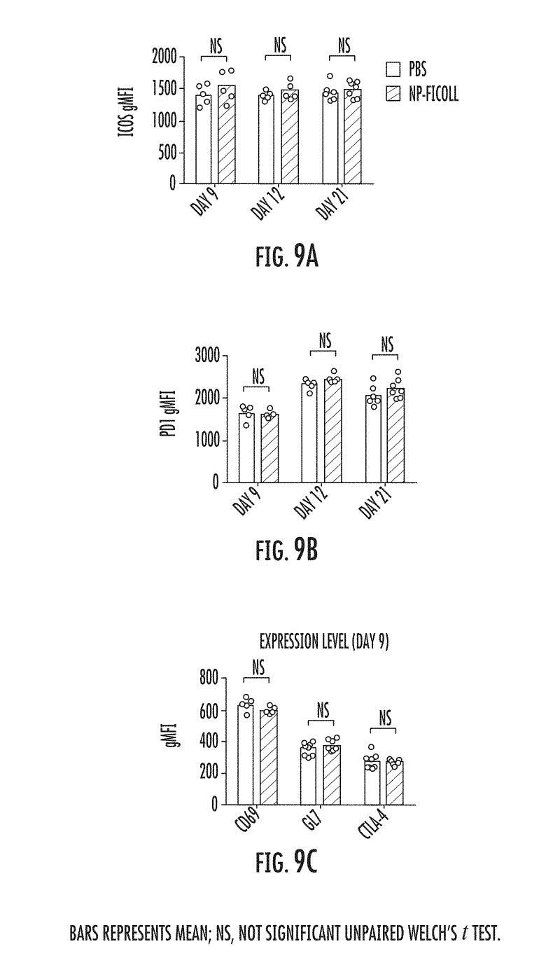

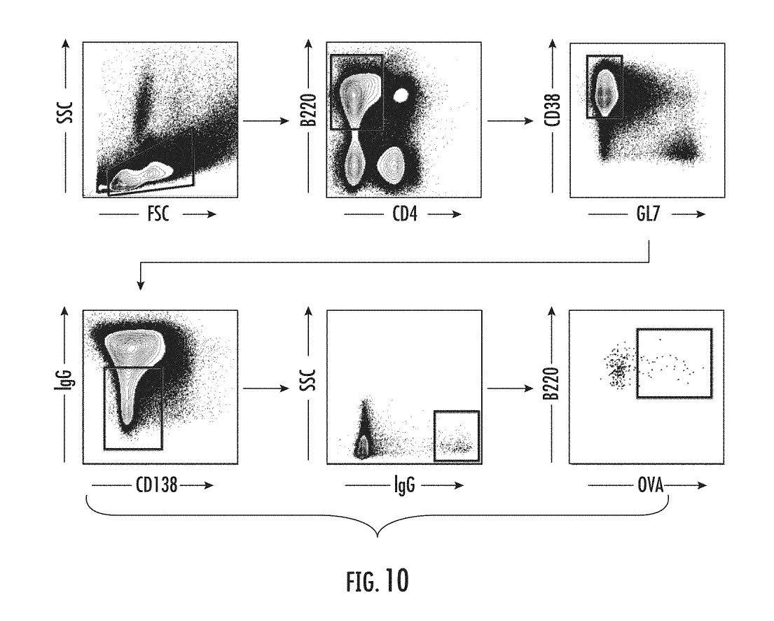

Mice were immunized and treated as in FIG. 4A. FIG. 5A: Gating strategy for total T follicular or Tfr cells (CD4⁺, CD19⁻, CXCR5^{hi}, PD1^{hi} and CD4⁺, CD19⁻, CXCR5^{hi}, PD1^{hi}, Foxp3^{-/+}). FIGS. 5B, 5C: Quantification of T follicular and T follicular regulatory (Tfr) cells. FIG. 5D: Ratio of T follicular to GC B cell after NP-Ficoll treatment on day 9. FIGS. 5E, 5F: Representative OVA-specific germinal center dark zone (DZ) gate (B220⁺, GL7⁺, CD4⁻, CD38^{lo}, OVA⁺, CxCr4^{hi}, CD86^{lo}) and quantification on day 9. FIGS. 5G, 5H) Representative staining intensity of pS6 and quantification. Bars represent mean; NS, not significant; *p<0.05, **p<0.01, unpaired Welch's t test.

FIGS. 6A-E show that attenuating GC competition increases subdominant long-lived plasma cell, memory B cell, and Ab production. Mice were immunized and treated as in FIG. 4A. FIG. 6A, 6B: NP-specific (A) and OVA-specific (B) ELISPOTs obtained from bone marrow 21 days after primary immunization. Spots are representative of three replicate wells per mice, 10 mice per group. FIGS. 6C, 6D: Serum anti-NP (C) and anti-OVA Ab (D) obtained at various time points. Bar represents mean+/−SEM, n=7 or 8 per group. FIGS. 6E, 6F: Representative flow plots of IgG1⁺ OVA-specific memory B cells (B220⁺, CD4⁻, CD38⁺, GL7⁻, CD138⁻, IgD^{lo/-}, IgG1⁺, OVA⁺) and quantification. FIG. 6G: α-OVA Ab recall response. Mice were allowed to rest for 21 weeks after immunization and were then challenged with 10 μg of NP-OVA w/o adjuvant. Data is pooled from 2 independent experiments representing change in Ab response from 1 day before challenge to 8 days post challenge for individual mice. Bars represent mean; *p<0.05, **p<0.01, ****p<0.0001, Welch's t test.

FIGS. 7A-C show that NP-OVA and OVA immunized mice have similar T follicular responses. FIG. 7A, FIG. 7B: Quantification of T follicular cells (A) and T follicular regulatory cells (B) CD4⁺, CD19⁻, CXCR5^{hi}, PD1^{hi} and CD4⁺, CD19⁻, CXCR5^{hi}, PD1^{hi} Foxp3⁺. FIG. 7C: Anti-OVA serum IgG Ab response quantified by ELISA on day 10 after immunization. Bars represent mean; NS, not significant; *p<0.05 unpaired student's t test.

FIGS. 8A-J show antigen specific reduction of GC B cells after treatment with inhibitory construct. FIGS. 8A, 8D: Mice were immunized and treated as in FIG. 4A. (A) Representative tunel staining of GCs 6 hr after NP Ficoll treatment (B) Quantification of tunel+ area in individual GCs of control on NP-Ficoll treated mice. (C) Representative gates of λ⁺ GC B cells (B220⁺, GL7⁺, CD4⁻, CD38^{lo}, λ⁺) and quantification (D). FIGS. 8E, 8F: Mice were immunized with unconjugated OVA in alum and treated with NP-Ficoll as in FIG. 4A. Quantification of total GC B cells (E) and OVA-specific GC B cell number (F) after NP-Ficoll treatment (day 9). FIGS. 8G, 8H: Mice were immunized i.p. with 1 μg of CRM-OVA then treated with soluble OVA or PBS on day 7. Spleens were dissected 12 days after immunization. (G) Quantification of total GC frequency and OVA specificity (H). FIGS. 8I, 8J: Representative flow plot of mice immunized with unconjugated CRM and quantification of GC specificity (day 12) (J). Bars represent mean; NS, not significant; **p<0.01, ***p<0.001, ****p<0.0001 unpaired Welch's t test.

FIGS. 9A-C show the T follicular cell activation status after NP-Ficoll treatment. FIGS. 9A-9C: Mice were immunized and treated as in FIG. 4A. T follicular cell CD4⁺, CD19⁻, CXCR5^{hi}, PD1^{hi} expression level of various activation markers at indicated time points. Bars represents mean; NS, not significant unpaired Welch's t test.

FIG. 10 is a flow chart for OVA-specific memory B cell gating. Mice were immunized and treated as in FIG. 4A. FIG. 10: Gating strategy for OVA+ memory B cells.

### DETAILED DESCRIPTION

The present invention provides vaccines, methods of immunizing and methods for generating broadly-neutralizing antibodies (bnAb).

Rapidly evolving pathogens such as HIV or influenza virus can rapidly mutate their antigenic profiles, thereby escaping conventional vaccines. However, epitopes that are essential for pathogen survival or infectivity, i.e., neutralizing epitopes, are often highly conserved among heterologous strains of the pathogens. These epitopes represent a key vulnerability that is targeted in the compositions and methods described herein. The antigenicity of these conserved epitopes is frequently subdominant. Hence, these conserved epitopes are not efficient at activating cognate B cells. During the humoral response, only a limited level of survival and propagation signals are available. As a result, B cells that are specific toward these broadly-neutralizing epitopes are outcompeted by immunodominant, non-broadly-neutralizing B cell clones. To address this issue, the methods and compositions of the invention provide selective elimination or suppression of immunodominant B cells during an active humoral immune response to allow expansion of subdominant B cells that bind to these neutralizing epitopes.

Disclosed herein are methods and compositions that eliminate (partially or completely) the immunodominant, non-broadly-neutralizing immune response. The invention enables preferable expansion of immuno-subdominant B cells that target specific epitopes of interest and may be applied to generate broadly neutralizing vaccines and antibodies against any elusive pathogen, such as influenza virus or HIV, for example.

In some embodiments of the invention, the methods and vaccines deliver inhibitory, apoptopic and/or tolerogenic signals (referred to herein as “inhibitory signals”) to B cells that recognize and bind to non-neutralizing epitopes of the pathogen of interest. In this way, the immune response is focused toward neutralizing epitopes of the pathogen, resulting in the generation of neutralizing antibodies. Inhibitory signals are delivered to non-neutralizing B cells via certain targeting attributes of the inhibitory construct.

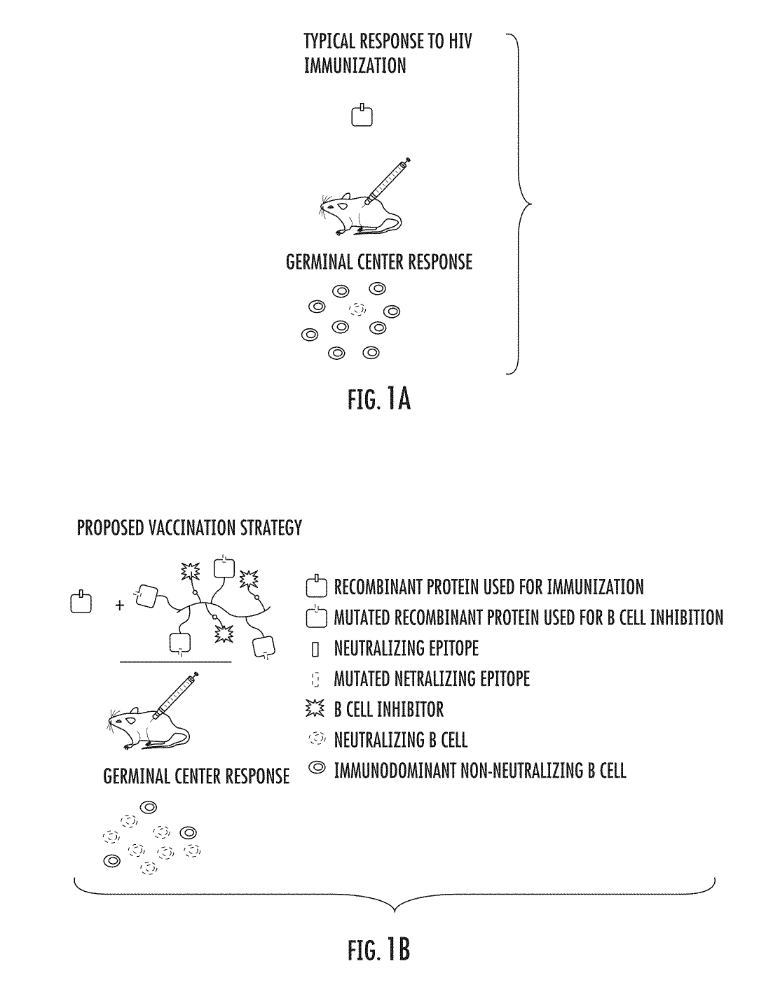

In one aspect, the invention provides a method for immunizing an individual against a pathogen by inhibiting the expansion of B cells that bind to non-neutralizing epitopes present on an antigen of the pathogen during an immune response and enhancing the expansion of broadly neutralizing B cells that bind to neutralizing epitopes present on an antigen. In general, interclonal competition inhibits full participation of subdominant B cells in the germinal center (GC). However, as shown herein, selective elimination or suppression of expansion of immunodominant B cells during an active germinal center response to a pathogen allows subdominant B cells (neutralizing B cells) to expand unimpeded. Without competition or with reduced competition, these selected subdominant B cells generate an improved long-lasting humoral immune response. FIGS. 1 A and B are schematic representations of a typical immunization and the developed immunization regimen of the invention, showing the differences in germinal center responses.

In another aspect of the invention, the disclosure provides vaccines for mammalian pathogens, which inhibit the expansion of B cells that bind to non-neutralizing epitopes present on an antigen of the pathogen and enhance the expansion of broadly neutralizing B cells that bind to neutralizing epitopes present on the antigen.

The present vaccines for and methods of suppressing the expansion of non-neutralizing B cells, enhancing the expansion of broadly neutralizing B cells and immunizing subjects against a pathogen are applicable to any subject, particularly mammalian subjects and preferably humans. In one aspect, immunization against a particular pathogen includes a regiment of (1) administering an effective amount of a primary immunization composition containing a recombinant peptide comprising a neutralizing epitope of the pathogen and (2) administering, either separately or together with or admixed with the primary immunization composition, a composition containing an effective amount of an inhibitory construct comprising an altered neutralizing epitope that cannot bind neutralizing antibody. In certain embodiments, the primary immunization composition comprises a recombinant protein naturally expressed by the pathogen, which includes a neutralizing epitope, and the inhibitory construct comprises at least one copy of a mutated or altered form of the recombinant peptide that comprises at least one alteration of the neutralizing epitope, which prevents antibody binding to the epitope. In other embodiments, the inhibitory construct is administered together with a molecule or compound that inhibits non-neutralizing B cells, causes apoptosis of non-neutralizing B cells or otherwise causes immunotolerance. The inhibitory molecule or compound is referred to herein as an “inhibitory signal.”

In other embodiments, the primary immunization composition comprises a peptide, such as a recombinant peptide comprising a neutralizing epitope and a non-neutralizing (i.e., immunodominant) epitope of the pathogen, and the inhibitory construct comprises a water-soluble form of the immunodominant, non-neutralizing epitope.

The primary immunization compositions used in the methods and vaccines of the invention include a recombinant peptide comprising at least a portion of a protein expressed by the pathogen for which immunization is desired and which contains a neutralizing epitope. The primary immunization composition contains a sufficient amount of the peptide or recombinant peptide to invoke a humoral immune response, such as for example, 50 to 150, mcg, preferably 75 to 100 mcg.

Neutralizing epitopes of many pathogens are known. Nonlimiting examples of known HIV neutralizing antibodies, for example, include:
(1) V2 trimer apex—a peptide containing regions of the V1-V2 domain and the V3 loop (consisting of a combination of but not exclusively, the amino acids between and including positions 165-171, and glycans N156 and N160). Examples of neutralizing antibodies that bind to the V2 trimer apex include PG9, PG16, CH01-04 and PGT140s (Wibmer et al., 2015).
(2) N332 supersite—a peptide containing regions on the V3-V4 domain (consisting of a combination of, but not exclusively the amino acids between and including position 323 and 330, and glycans N137, N156, N295, N301, N332, N334, N339, N386, N392). Examples of neutralizing antibodies that bind to the N332 supersite include PTG120s, PTG130s, 2G12 (Wibmer et al., 2015).
(3) CD4 binding site (CD4bs)—a peptide including regions of the CD4 binding loop, Loop D and beta-23-loop V5 (consisting of a combination of, but not exclusively the amino acids between and including position 275-283, 362-374, 455, 467 and glycans N156 and N160). Examples of neutralizing antibodies that bind to the CD4bs include 2F5, Z133e1, 4E10 and 10E8 (Wibmer et al., 2015).

Other HIV neutralizing epitopes are disclosed in the HIV Databases, Zhou et al., Immunity, 39:245-258, Aug. 22, 2013; and Scheid et al., Science, 33:1633-1637, Sep. 16, 2011, for example, and are incorporated herein by reference. One with ordinary skill in the art can readily identify neutralizing epitopes utilizing these resources.

Neutralizing epitopes of infectious diseases can readily be identified by a person of ordinary skill in the art by searching free databases such as the Immune Epitope Database and Analysis Resource and are incorporated herein by reference.

In certain embodiments, the inhibitory construct is separately delivered from the primary immunization composition, e.g., prior to, concurrently with or following administration of the primary immunization composition and in other embodiments, is included in the primary immunization composition. The inhibitory construct comprises the peptide or recombinant peptide used for the primary immunization except that the neutralizing epitope of the peptide or recombinant peptide is altered in a manner that prevents binding of neutralizing antibodies thereto. Such alterations include for example, one or more amino acid changes (mutations) of the epitope sequence or the removal, addition, or alteration of the glycan structure of the epitope (the glycosylation pattern, number of monosaccharides attached to the epitope, and the like) that eliminate or reduce antibody binding to the epitope. Methods for adding or removing glycans are known to those of skill in the art and include, for example, adding or altering the glycosylation consensus sequence (asparagine—X—serine/threonine where x is any amino acid except proline). Methods of altering glycan structure of proteins are known to those of skill in the art and include for example, enzymatic digestion of the peptide with PNGase F, for example, or altering the glycosylation processes during protein synthesis, such as by treating with kifunensine. One of skill in the art can readily determine whether a particular alteration(s) of the neutralizing epitope eliminates or reduces antibody binding to the epitope.

In some embodiments, the inhibitory construct is administered with an inhibitory signal. Non-limiting examples of inhibitory signals include cyclophosphamide, methotrexate, doxorubicin, aldoxorubicin, cisplatin, a BCL-6 inhibitor, BCL-2 inhibitor, mtor inhibitor, MEK inhibitor, CD22 ligand or a FAS ligand.

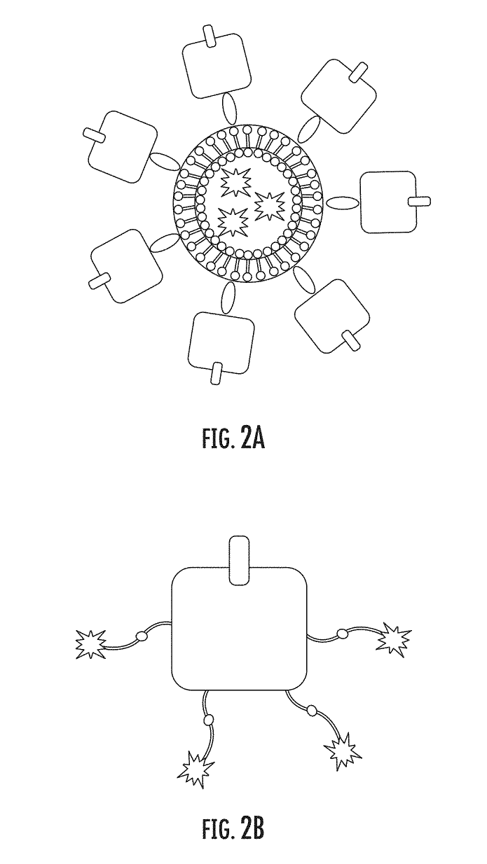

The inhibitory construct and/or inhibitory signal may be delivered by any means known for delivering therapeutic compositions. For example, a liposomal delivery system may be used in which the inhibitory construct is displayed on the surface of a liposome, while an inhibitory signal is incorporated within the liposome core or within the liposome bilayer (Sercombe et al., 2015) (FIG. 2A) Alternatively, the inhibitory signal may be conjugated to the liposomal surface. (FIG. 2B)

Other methods of delivery of the inhibitory construct and inhibitory signal include use of a nanocage-based delivery system in which the inhibitory construct is chemically linked to the surface of a protein nanocage. Non-limiting examples of protein nanocages include lumazine synthase, ferritin, Qbeta, and the virus-like particles CMV, MS2, CPMV and CCMV (Lee et al., 2016). Protein nanocages are self-assembling structures that perform biological functions in living cells such as iron storage and prevention of DNA oxidative damage. Their size of ˜15 to ˜100 nm and serum stability make them ideal candidates for drug and antigen delivery through lymphatics. In this embodiment, the inhibitory signal may either be chemically tethered to the protein nanocage or incorporated into the nanocage core via electrostatic or hydrophobic interactions. The inhibitory construct may either be chemically tethered to the surface of the protein nanocage or be genetically engineered to be displayed on the surface of said nanocage via a peptide linker attached to either the N or C terminus. Recombinant versions of protein nano-cages can be readily produced by persons with ordinary skill in the art (Jardine et al., 2013) U.S. Pat. No. 7,709,010B2; WO2004056389A1; (Min et al., 2014); each incorporated in their entireties by reference thereto).

In other embodiments, the inhibitory construct may be delivered using a polymer based delivery system in which the inhibitory construct and inhibitory signal are each conjugated to a biocompatible polymer such as polyethylene glycol, Ficoll, dextran, or N-(2-hydroxypropyl)methacrylamide (Lynn et al., 2015) Alternatively, the inhibitory signal may be directly conjugated to the inhibitory construct via one or more surface exposed amine, sulfhydryl, or carboxyl functional groups present on the inhibitory construct. (FIG. 2B) In other embodiments, administration of the inhibitory construct alone is sufficient to induce immune tolerance. In certain embodiments, primary immunization compositions may include, in addition to the recombinant peptide containing a neutralizing epitope, one or more immunostimulants. An immunostimulant refers to essentially any substance that enhances or potentiates an immune response (antibody or cell-mediated) to an exogenous antigen. One preferred type of immunostimulant is an adjuvant.

Many adjuvants contain a substance designed to protect the antigen from rapid catabolism, such as aluminum hydroxide or mineral oil, and a stimulator of immune responses, such as lipid A, Bordetella pertussis or Mycobacterium tuberculosis derived proteins. The adjuvant may be a submicron oil-in-water emulsion of a metabolizable oil and an emulsifying agent. For example, the adjuvant may comprise MF59™, which is a sub-micron oil-in-water emulsion of a squalene, polyoxyethylene sorbitan monooleate (Tween™ 80) and sorbitan trioleate. The adjuvant may also be a combination of the TLR4 agonist MPL (3-O-desacyl-4′-monophosphoryl lipid A) and aluminum salt, e.g., ASO4 (GlaxoSmithKline, Philadelphia, Pa.).

Certain adjuvants are commercially available as, for example, Freund's Incomplete Adjuvant and Complete Adjuvant (Difco Laboratories, Detroit, Mich.); Merck Adjuvant 65 (Merck and Company, Rahway, N.J.); ASO1, ASO2, ASO3, and ASO4 (GlaxoSmithKline, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

The primary immunization and inhibitory compositions may include a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the recombinant peptide and/or the inhibitory composition and to minimize any adverse side effects in the subject, as would be well known to one of skill in the art.

The materials of the primary immunization composition and inhibitory composition may be in solution, suspension (for example, incorporated into microparticles, liposomes, or cells). Suitable carriers and their formulations are described in Remington: The Science and Practice of Pharmacy (22nd ed.) eds. Loyd V. Allen, Jr., et al., Pharmaceutical Press, 2012. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5.

Pharmaceutical carriers are known to those skilled in the art. These most typically would be standard carriers for administration of vaccines to humans, including solutions such as sterile water, saline, and buffered solutions at physiological pH. Pharmaceutical compositions may include carriers, thickeners, diluents, buffers, preservatives, surface active agents and the like in addition to the recombinant peptide and any adjuvant.

A method of immunizing a subject against a particular pathogen involves administering the disclosed primary immunization composition and composition containing the inhibitory construct (the inhibitory composition) to a subject in need thereof. The primary immunization composition and inhibitory composition may be administered in a number of ways. For example, each composition may be separately and independently administered orally, intravenously, subcutaneously, transdermally (e.g., by microneedle), intraperitoneally, ophthalmically, vaginally, rectally, sublingually, or by inhalation. Preferably, the primary immunization composition and inhibitory composition are administered intramuscularly or subcutaneously.

The exact amount of the compositions required will vary from subject to subject, depending on the species, age, weight and general condition of the subject, the mode of administration and the like. Thus, it is not meaningful to specify an exact amount for every composition. However, an appropriate amount of each composition can be determined by one of ordinary skill in the art using only routine experimentation given the teachings herein. For example, effective dosages and schedules for administering each of the compositions of the vaccine may be determined empirically, and making such determinations is within the skill in the art. The dosage ranges for the administration of the compositions are those large enough to produce the desired immune response. The dosage of each of the primary immunization composition and inhibitory composition should not be so large as to cause adverse side effects, such as unwanted cross-reactions, anaphylactic reactions, and the like. The dosage can be adjusted by the individual physician in the event of any counterindications. Of each of the primary immunization composition and inhibitory composition can vary, and each can be administered in one or more dose administrations daily, for one or several days. Guidance can be found in the literature for appropriate dosages for given classes of pharmaceutical products.

The immunization regimen may include a single administration of a dosage form of the primary immunization composition (the “primary immunization”), followed by administration of the inhibitory composition. Preferably, the inhibitory composition is administered from one to 30 days following primary immunization. In certain embodiments, the inhibitory composition is administered within two months of primary immunization. Administration of the inhibitory composition may include a once daily administration of the inhibitory composition for one day to 30 days, for a total of from 1 to 30 doses of inhibitory composition following administration of the primary immunization composition.

In other embodiments, the inhibitory composition is administered prior to administration of the primary immunization composition. For example, the inhibitory composition may be delivered once daily from one to 30 days prior to primary immunization for a total of from 1 to 30 doses. For example, a first dose of the inhibitory composition may be administered at any time within two months of administration of the primary immunization composition.

In other embodiments, the inhibitory composition is administered along with administration of the primary immunization composition, as either a separate composition admixed with the primary immunization composition.

There may also be a gap in time between primary immunization and the onset of inhibitory composition administration, regardless of whether the inhibitory composition is administered before or after primary immunization. For example, the inhibitory composition may be administered at any time, once daily within the first two months of administration of the primary immunization composition.

The present rational epitope targeting strategy can be applied to any vaccine or vaccination regimen where a targeted antibody response is desired. For example, the immune response can be targeted against such pathogens as HIV, influenza virus, shigella, herpes virus, plasmodium species, dengue, mycobacterium tuberculosis, cytomegalovirus, respiratory syncytial virus, ebola virus, hepatitis C, West Nile virus, streptococcus pneumoniae, streptococcus aureus, and streptococcus pyrogenes. Several antigen databases that enable identification of epitopes that are immune-subdominant or immune-dominant are available.

Also, genes encoding putative conserved antigenic epitopes (neutralizing epitopes) can be bioinformatically identified, for example, using a genomic sequence resource for the pathogen of interest based on all or some of the following criteria: possession of a signal peptide (SP), one or more transmembrane domains (TMD) or a glycosylphosphatidylinositol (GPI) anchor, an elevated ratio of non-synonymous to synonymous substitutions (dN/ds) between orthologous genes in the genomes of different species of the pathogen, if appropriate. Expressed sequence tag (EST) data may also be used to evaluate stage-specific expression of each candidate gene, if appropriate. Epitope analysis tools provided by the Immune Epitope Database and Analysis Resource (iedb.org) can also be used to identify epitope conservation across pathogen strains and may be useful in identifying epitopes with potential for broad neutralization.

Bioinformatically identified candidate genes may be cloned, using known methodologies such as the Gateway® (Invitrogen™) in vitro recombination method [Betton J M (2004) High throughput cloning and expression strategies for protein production. Biochimie 86: 601-605], for example, and expressed as recombinant protein. The recombinant peptides are screened for immunogenicity through animal immunization, for example and effective candidate peptides may be included in the primary immunization composition and altered as described above for use in the inhibitory composition component of the vaccine.

In some embodiments antibody generating non-human vertebrates such as rats and mice are used to develop antibodies bearing human variable regions for research and/or therapeutic use. Methods for producing therapeutic human sequence antibodies with use of transgenic animals are known in the art. Examples include U.S. Pat. No. 6,130,364 U.S. Pat. No. 7,501,552, U.S. Pat. No. 6,673,986, U.S. Pat. No. 9,796,788B2, U.S. Pat. No. 9,788,534B2, U.S. Pat. No. 9,622,459B2, U.S. Pat. No. 9,346,873B2, U.S. Pat. No. 9,204,624B2, US20080196112A1, US20090255002A1, GB2501753A, WO2011004192, WO2013144567A1, WO2013079953A1, the disclosures of which are incorporated by reference in their entirety. Current strategies for developing therapeutic monoclonal antibodies require extensive screening to isolate B cells that produce antibodies with desired functionalities. The rational epitope targeting strategy described herein significantly reduces screening time and provides a method to create antibodies against epitopes which would otherwise not develop naturally (i.e. immune-subdominant epitopes). For example, the present methods can be used to target specific epitopes of receptors, cytokines, enzymes, enzyme substrates, hormones, or any other biologically active ligand to create an agonist, antagonist, or other functional effect.

In certain embodiments, the method of generating therapeutic antibodies to a specific epitope of a therapeutic target comprises the steps of:
(1) administering a primary immunization composition to a mammal, such as a transgenic animal, said primary immunization composition comprising a recombinant peptide comprising the epitope of the therapeutic target;
(2) administering to the subject a composition comprising an inhibitory construct comprising at least one copy of an altered form of the recombinant peptide, wherein the altered form comprises at least one amino acid change or structural alteration of the epitope that prevents binding of antibody thereto; and wherein the inhibitory construct is administered separately from and prior to, concurrently with or within two months of administration of the primary immunization composition;
(3) Isolating one or more B cells from said mammal; wherein an antibody expressed by the one or more B cells comprises a variable domain and said antibody binds to the epitope; and
(4) Identifying the nucleic acid sequence that encodes the variable domain of said antibody and utilizing said sequence to generate a recombinant version of the antibody.
   A person with ordinary skill in the art can readily isolate antigen specific B cells, identify the nucleic acid sequence that encodes the variable domain and generate recombinant antibodies, for example, using involves single cell sorting via fluorescence activated cell sorting (FACS) and V(D)J transcript amplification by RT-PCR. Gene fragments are then recombined into expression vectors followed by transfections into a protein-producing cell line such as HEK293. (Starkie et al., 2016; Tiller et al., 2008; von Boehmer et al., 2016).

A number of embodiments of the invention have been described. Nevertheless, it will be understood that various modifications may be made without departing from the spirit and scope of the invention.

### EXAMPLES

### Example 1

### Experimental Procedures Used in Examples 2-4

### Mice, Immunizations, and Treatments

Animal work was in accordance with Institutional Animal Care and Use Committee at Northeastern University. C57BL/6J mice were purchased from The Jackson Laboratory (Bar Harbor, Me.) and held under specific pathogen-free conditions. Age and sex matched mice between 7 and 10 weeks of age were used for all experiments. Four to ten mice per group were used in each analytical experiment. NP-OVA was conjugated in-house at a molar ratio of 4:1 NP:OVA. NP-OSu was purchased from Biosearch technologies (N-1010-100) and ovalbumin was obtained from Sigma Aldrich (A5503). Unconjugated NP hapten was removed using Amicon Ultra 30K centrifugal filter units (Millipore). CRM197 was purchased from Scarab Genomics and was conjugated to ovalbumin at a 1:1 molar ratio using Pierce Controlled Protein-Protein Crosslinking kit (Thermo Scientific) following the manufacturer's instructions. Mice were immunized with 10 μg of NP-OVA i.p. in precipitated alum as described in (Cain et al., 2013) (Cain, Sanders, Cunningham, & Kelsoe, 2013). NP-Ficoll was purchased from Biosearch Technologies (F-1420-100). Mice were treated with daily i.v. injections of 500 μg of NP₁₈-Ficoll or NP₃₀-Ficoll from days 6-8 following immunization. For memory recall experiments, mice were immunized with NP-OVA and treated with NP-Ficoll as stated above and allowed to rest for 21 weeks. 10 μg of NP-OVA without adjuvant was administered i.v. and serum was collected 8 days later for analysis. In some experiments mice were immunized with 1 μg of CRM-OVA in alum i.p. then treated with 4 mg of soluble OVA on day 7 along with 150 ug of isoproterenol (Millipore) as previously described (Han and Kelsoe, 1995).

### Flow Cytometry

Spleens were forced through a 70 μm strainer into PBS supplemented with 5% FCS. RBCs were lysed with ACK buffer (Gilbco) followed by 10 min incubation with 1 μg/ml Fc Block (24G2; BD biosciences) then stained for 30 min at 4° C. with the following antibodies acquired from Biolegend or BD Biosciences: B220 (RA3-6B2), CD138 (281-2), CD19 (ID3), CD38 (90), CD4 (RM4-5), CD86 (GL-1), CXCR5 (2G8), CXCR4 (2B11), Foxp3 (MF-23), GL-7 (GL7), ICOS (7E.17G9), IgD (11-26c.2a), IgG1 (A85-1), PD-1 (J43). Antigen specific GC responses were detected with Alexa Fluor 647 tagged OVA (Thermo Fisher), NP-PE (Biosearch Technologies), or with FITC tagged CRM (made in house) amplified by biotinylated α-FITC (Biolegend) and BB515 Streptavidin (BD Biosciences). Phospho S6 (pS6) staining was performed as described in (Ersching et al., 2017). For intracellular staining, samples were fixed and permeabilized prior to staining (Foxp3/transcription factor buffer set; eBioscience). Acquisition was conducted on a Cytek DxP11 FACS Calibur and analyzed on FlowJo X (Tree Star).

### Immunohistochemistry

Spleens from immunized mice were frozen in Tissue-Tek compound in a liquid nitrogen-cooled bath of 2-methylbutane. 5-μm sections were cut on a cryostat, air-dried then fixed in ice-cold acetone for 10 min. Sections were rehydrated (0.5% BSA, 0.1% Tween 20 in PBS), FC-blocked and stained with TUNEL apoptosis kit (Invitrogen C10619) following the manufacturer's instructions. Sections were then stained with PNA-FITC (Vectorlabs) and B220 (RA3-6B2) for 1 hr at RT. Images were acquired on a Zeiss LSM 710 microscope and ImageJ/Fiji was used for analysis. Briefly, mask files were created for GCs (PNA⁺, B220⁺ area) and apoptotic nuclei (TUNEL⁺). Relative apoptosis was quantified based on percent TUNEL⁺ area within individual GC area.

### ELISA and ELISPOT

For ELISA, 96-well plates (2595; Costar) were coated with 2 μg/ml NP₃₀-BSA, or 10 μg/ml OVA (Biosearch Technologies and Sigma-Aldrich) in PBS overnight. Plates were washed twice (0.5% BSA, 0.1% Tween 20 in PBS), blocked for 1 h (0.5% BSA in PBS), washed twice, and serially diluted samples were added for 1 h RT. Plates were washed three times and HRP conjugated detection Abs for IgG (Bethyl Laboratories) were added for 1 h, washed three times, and tetramethylbenzidine substrate was added (BD Biosciences). Reaction was stopped using 2 N H2SO4 and read at 450 nm. Ab quantification was calculated based on NP or OVA specific monoclonal standards (H331γ1 or OVA-14) and reported as relative units (RU). For ELISPOTs, Immobilon-P plates (Millipore) were coated overnight at 4° C. with 2 μg/ml NP₃₀-BSA or 10 μg/ml of OVA in PBS. Plates were washed twice then blocked for 2 h. BM cells were isolated, RBC lysed with ACK, then incubated for 3 h at 37° C. in RPMI. The plates were washed three times then incubated with alkaline phosphatase-conjugated Abs to IgG (SouthernBiotech). Plates were washed five times and developed using NBT reagent (Sigma-Aldrich).

### Statistical Analyses

All statistical analyses were performed using GraphPad® prism. Statistical significance was determined using a two-tailed unpaired Welch's t test. P values of less than 0.05 were considered significant.

### Example 2

### NP-OVA as a Model for Interclonal Competition in the Germinal Center

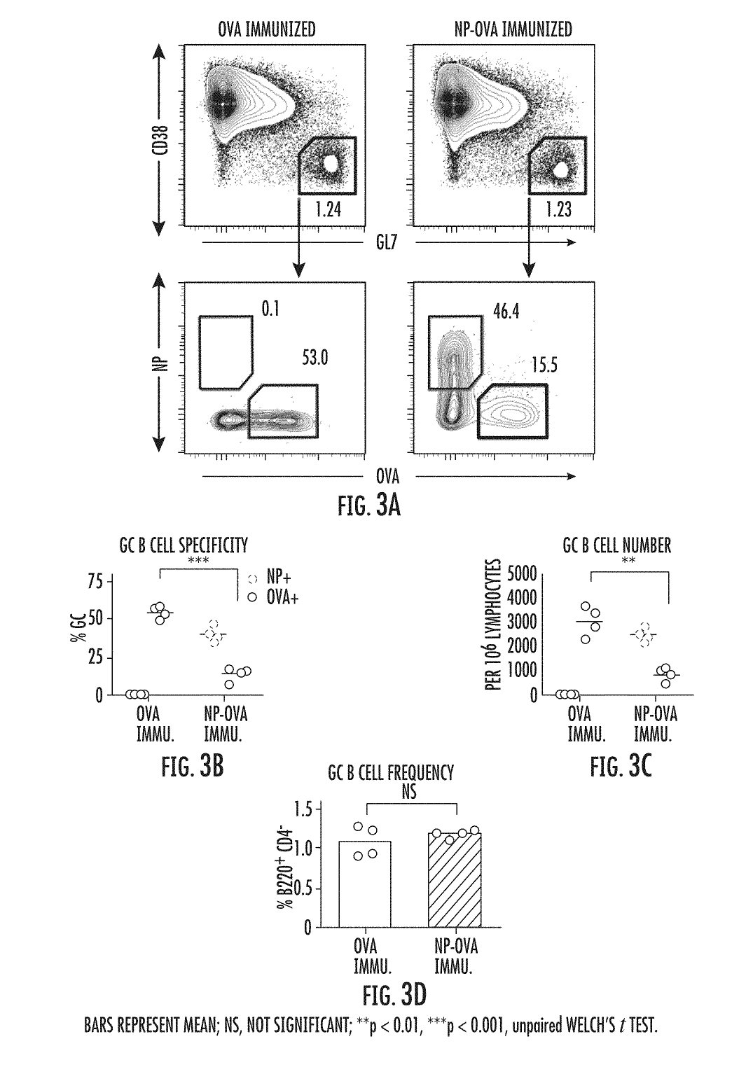

NP-OVA contains two discrete antigenic moieties, the NP epitope and the polyepitopic OVA carrier protein, for which an antigen-specific GC response can be easily analyzed by fluorescently conjugated NP-Phycoerytherin or OVA-Alexa Fluor 647 (FIG. 3A). Immunization with NP-OVA consistently generated a GC response where NP-specific cells comprised a three-fold greater proportion of the GC as opposed to OVA (40 vs. 14%, respectively; FIGS. 3A-3C).

To test whether interclonal competition affects the participation of subdominant cells in, C57BL/6J mice were immunized intraperitoneally (i.p.) with 10 μg of either NP-OVA or unconjugated OVA in precipitated alum. The mice were sacrificed 12 days after immunization and spleens were harvested for analysis by flow cytometry. Both OVA and NP-OVA immunized groups generated comparable GC responses (FIGS. 3A and 3D) as well as T follicular and T follicular regulatory (Tfr) responses (FIGS. 7A and 71B, respectively). As expected, the presence of the immunodominant NP epitope significantly reduced the proportion of OVA-specific cells (14% NP-OVA immunized vs. 55% in OVA immunized; FIGS. 3A-1C). Serum anti-OVA IgG Ab response was also reduced by the presence of the NP epitope (FIG. 7C).

Consistent with recent reports that GCs are capable of maintaining a considerable level of clonal diversity (Kuraoka et al., 2016; Tas et al., 2016), these data show that despite the dominance of NP⁺ cells, subdominant OVA⁺ B cells still comprise a substantial portion of the GC during the immune response. These data demonstrate that interclonal competition reduces the proportion of subdominant B cell clones that participate in the GC reaction. In addition, the data show that absent this competition, OVA-specific GC B cells are intrinsically capable of populating a large percentage (>50%) of the GC.

### Example 3

### Soluble Antigen Treatment Reduces the Number of Immunodominant B Cells in Germinal Centers and Favors the Expansion of Subdominant Cells

Two groups of mice were immunized with 10 μg NP-OVA, then treated with daily intravenous (i.v.) injections of either NP-Ficoll or PBS during the early GC response (days 6-8; FIG. 4A). The effect of NP-Ficoll on GC B cell frequency and specificity was assessed at 9, 12, and 21 days after immunization (1, 4, and 13 days after NP-Ficoll treatment). NP-specific GC B cells, along with total GC B cell frequency, were significantly reduced shortly following soluble antigen administration indicating GC B cell apoptosis (day 9; FIGS. 4B, 4C, 8A, 8B). Consistent with previous reports (Han and Kelsoe, 1995; Pulendran et al., 1995), this effect was observed to be antigen specific as NP-Ficoll administration to mice immunized with unconjugated OVA did not affect either total GC or OVA-specific B cell counts (FIGS. 8E and 8F). Additionally, this effect cannot be attributed to masking of the NP-specific BCR by NP-Ficoll as the prototypical NP-specific, λ1 light-chain positive, B cells are also reduced following treatment (FIGS. 8C and 8D) (Jacob and Kelsoe, 1993). Although the total GC frequency quickly recovered to control levels by day 12, the NP response, as measured by percent GC and cell number, was persistently reduced throughout the remainder of the GC response (through day 21; FIGS. 4D, 2E). In contrast, NP-Ficoll did not affect subdominant OVA-specific GC cell numbers, therefore they encompassed a greater proportion of the smaller GC on day 9 (FIGS. 4D-2F). Importantly, as the GC response progressed, OVA⁺ cells expanded significantly after elimination of NP⁺ cells. Three days after the final soluble dose (day 12), OVA⁺ cells increased by approximately 2 fold, as shown by both GC percentage and cell number, and remained elevated until the conclusion of the study (day 21; FIGS. 4D and 4F). Taken together, these data show that reducing interclonal competition during the early GC response significantly diminishes the selective pressure imposed on subdominant B cells and allows these cells to expand and encompass a greater proportion of the GC for the remainder of the response.

Because the GC response to haptens can be an oversimplified representation of events occurring following protein immunization or microbial infection (Kuraoka et al., 2016; Tas et al., 2016), validation of these results using a more relevant complex antigen was undertaken. For these studies, a protein-protein conjugate of CRM197 (non-toxic mutant of diphtheria toxin) and OVA at a 1:1 molar ratio was developed for use as the immunizing antigen. Compared to mice immunized with unconjugated CRM197, CRM-OVA immunized mice had a substantially weaker response to CRM, indicating that the OVA domain is immunodominant in this setting (FIGS. 8G left, 8I and 8J).

To determine whether eliminating immunodominant B cells during an active GC reaction could shift the response toward the subdominant CRM domain similarly to what had been observed in the previous experiments, two groups of mice were immunized with 1 μg CRM-OVA, then treated with one i.p. injection of either 4 mg of OVA or PBS on day 7 post immunization. The effect of soluble OVA on GC B cell frequency and specificity was assessed on day 12. Mice treated with soluble OVA had a significant reduction in OVA-specific GC B cells (FIGS. 4G and 8H). Concurrently, OVA treated mice had a significant increase in CRM-specific cells as assessed by both percent GC as well as cell count (FIG. 4H). As with the previous experiments, total GC quantity was not affected at this time point (FIG. 8G). These data show that the soluble antigen strategy can skew the response to specific domains within a protein antigen and guide the humoral response toward sought-after, conserved regions of microbial proteins.

### NP-Ficoll Treatment Shifts OVA⁺ GC B Cell Population to Dark Zone

T follicular cells are critical to the development and maintenance of the GC reaction (Nutt and Tarlinton, 2011), Accordingly, the effect of soluble antigen on this population was investigated. No major perturbations in total T follicular and Tfr frequency in NP-Ficoll treated mice (FIGS. 5A, 5B, 5C). Their activation status (as measured by ICOS, PD1, CD69, GL7, and CTLA4 expression) also appears equivalent to control mice at all time points tested (FIG. 9A-C). As the GC B cell frequency is reduced by 48% shortly after soluble treatment (day 9; FIG. 4B, 4C), the largely unchanged T follicular population effectively creates an environment with significantly increased T follicular to GC B cell ratio (FIG. 5D). T follicular cell help has been shown to govern B cell residence time in the dark zone (DZ) of GCs and are thought to ultimately control their proliferative capacity (Gitlin et al., 2015; Gitlin et al., 2014). Consistent with previous reports, these data suggest that the increased level of T cell help provided in soluble antigen treated mice shifted the remaining OVA-specific cells to the DZ of the GC (Victora et al., 2010) and supported the expansion of this population (FIGS. 5E, 5F and 4F). Moreover, OVA-specific GC B cells from NP-Ficoll treated mice had increased mTOR activity (as measured by phosphorylated ribosomal protein S6; FIGS. 5G and 5H) which is indicative that these cells received stronger T cell help and were primed to undergo sustained proliferation in the DZ (Ersching et al., 2017). The data show that by eliminating the dominant B cell response, NP-Ficoll treatment appears to create an environment that is supportive for the remaining OVA⁺ GC B cells.

### Example 4

### Attenuating GC Competition Increases Subdominant Long-Lived Plasma Cell, Memory B Cell, and Ab Production

The GC reaction is the major source of a long-lived humoral response (Good-Jacobson and Shlomchik, 2010). Thus, the effects of the GC specificity changes imposed by the treatment described above translates to downstream functional effects. After 21 days following immunization, bone marrow (BM) cells were isolated and cultured to assess the extent of NP⁺ and OVA⁺ plasma cell (PC) formation. ELISPOT analysis demonstrated that elimination of NP⁺ cells during the early GC response translates to a reduction in NP-specific PC formation (FIG. 6A). Importantly, the frequency of OVA-specific PC in BM was considerably higher following NP-Ficoll treatment (FIG. 6B), in excellent correlation with the observation that the GC response was redirected toward the OVA epitope. Consistent with these results, the long-term IgG Ab response was significantly affected by the manipulations imposed on the early GC reaction. OVA-specific Ab in NP-Ficoll treated mice were markedly higher from day 35 through the remainder of the assay (day 63; FIG. 6D). The OVA Ab response in treated mice peaked long after the control group (day 49 vs. day 21) corroborating the increase in BM PC counts. Conversely, the NP-specific Ab response was significantly decreased during the late humoral response (day 35 through day 63; FIG. 6C). This late difference in Ab response is consistent with previous reports demonstrating that long-lived plasma cells are primarily generated during the late stages of the GC response (Weisel et al., 2016) and is also indicative that soluble antigen treatment is predominantly affecting the GC response, but not the extrafollicular response.

An analysis of how the immunization protocol affected the development of the immune-subdominant population was undertaken. In agreement with the GC and PC data, NP-Ficoll-treated mice developed significantly higher IgG1-switched OVA⁺ memory phenotype B cells at both time points tested (FIGS. 6E, 6F, 10). Additionally, NP-Ficoll-treated mice showed a robust increase in α-OVA Ab production upon antigen challenge 21 weeks after primary immunization while nearly all control mice failed to respond (FIG. 6G).

What is claimed is:

1. A method of immunizing a mammalian subject against a pathogen comprising the steps of:
(1) administering a primary immunization composition to the subject, said primary immunization composition comprising a peptide expressed by the pathogen, wherein said peptide comprises a neutralizing epitope; and
(2) administering to the subject a composition comprising an inhibitory construct comprising at least one copy of an altered form of the peptide, wherein the altered form comprises at least one amino acid change or structural alteration of the neutralizing epitope that prevents binding of neutralizing antibody thereto; and
wherein the inhibitory construct is administered prior to, concurrently with or within two months of administration of the primary immunization composition.

2. The method of claim 1, wherein the pathogen is HIV, Influenza Virus, or a Plasmodium species.

3. The method of claim 1, wherein the altered form of the peptide comprises one or more amino acid changes to the sequence of the neutralizing epitope.

4. The method of claim 1, wherein the altered form of the peptide comprises a structural alteration of the neutralizing epitope.

5. The method of claim 1, wherein the structural alteration of the neutralizing epitope comprises an alteration of the glycans attached thereto.

6. The method of claim 1, wherein the structural alteration of the neutralizing epitope is an addition to or removal of glycosylation sites attached to the neutralizing epitope.

7. The method of claim 1, wherein the neutralizing epitope is a broadly neutralizing epitope.

8. The method of claim 1, wherein the primary immunization composition and the composition comprising the inhibitory construct comprise a single composition.

9. The method of claim 1, wherein the primary immunization composition and the composition comprising the inhibitory construct comprise separate compositions.

10. The method of claim 1, wherein the inhibitory construct is administered once daily for one to 30 days following administration of the primary immunization composition.

11. The method of claim 1, wherein a first dose of the inhibitory construct is administered within two months of administration of the primary immunization composition.

12. The method of claim 1, wherein the inhibitory construct is administered together with an inhibitory signal.

13. The method of claim 12, wherein the inhibitory signal is selected from the group consisting of cyclophosphamide, methotrexate, doxorubicin, aldoxorubicin, cisplatin, a BCL-6 inhibitor, BCL-2 inhibitor, mtor inhibitor, MEK inhibitor, CD22 ligand and FAS ligand.

14. The method of claim 12, wherein the inhibitory construct is conjugated to an inhibitory molecule that inhibits B cell differentiation into germinal center (GC) B cells, plasma cells or memory B cells to form a peptide-inhibitory-conjugate.

15. The method of claim 12, wherein the inhibitory construct and the inhibitory signal are administered via a liposome comprising a core and a lipid bilayer, wherein the inhibitory signal is incorporated within the liposome core or lipid bilayer and the inhibitory construct is attached to an outer surface of the liposome.

16. The method of claim 12, wherein the inhibitory construct and inhibitory signal are each chemically linked to a nanocage-based delivery system.

17. The method of claim 12, wherein the inhibitory signal is conjugated to the inhibitory construct via an amine, sulfhydryl or carboxyl functional group present on the inhibitory construct.

18. The method of claim 1, wherein the inhibitory construct is a water-soluble peptide.

19. A method of generating therapeutic antibodies to a specific epitope of a therapeutic target, comprising the steps of:
(1) administering a primary immunization composition to a mammal, said primary immunization composition comprising a recombinant peptide comprising the epitope of the therapeutic target;
(2) administering to the subject a composition comprising an inhibitory construct comprising at least one copy of an altered form of the recombinant peptide, wherein the altered form comprises at least one amino acid change or structural alteration of the epitope that prevents binding of antibody thereto; and wherein the inhibitory construct is administered separately from and prior to, concurrently with or within two months of administration of the primary immunization composition;
(3) Isolating one or more B cells from said mammal;
wherein an antibody expressed by the one or more B cells comprises a variable domain and said antibody binds to the epitope; and
(4) Identifying the nucleic acid sequence that encodes the variable domain of said antibody and utilizing said sequence to generate a recombinant version of the antibody.

20. The method of claim 19 where the therapeutic target is selected from the group consisting of a cytokine, an enzyme, a chemokine, a hormone, a receptor, and a receptor ligand.

21. The method of claim 19 wherein the mammal is a transgenic animal that contains a fully human immunoglobulin repertoire.

22. The method of claim 19, wherein the altered form of the inhibitory construct comprises one or more amino acid changes to the epitope of interest.

23. The method of claim 19, wherein the structural alteration of the epitope is an alteration of the glycans attached thereto.

24. The method of claim 19, wherein the structural alteration of the epitope is an addition or removal glycosylation sites to the epitope of interest.

25. The method of claim 19, wherein the inhibitory construct is administered once daily for one to 30 days following administration of the primary immunization composition.

26. The method of claim 19, wherein a first dose of the inhibitory construct is administered within two months of administration of the primary immunization composition.

27. The method of claim 19, wherein the inhibitory construct is administered together with an inhibitory signal.

28. The method of claim 27, wherein the inhibitory signal is selected from the group consisting of cyclophosphamide, methotrexate, doxorubicin, aldoxorubicin, cisplatin, a BCL-6 inhibitor, BCL-2 inhibitor, mtor inhibitor, MEK inhibitor, CD22 ligand and FAS ligand.

29. The method of claim 27, wherein the inhibitory construct is conjugated to an inhibitory molecule that inhibits B cell differentiation into germinal center (GC) B cells, plasma cells or memory B cells to form a peptide-inhibitory-conjugate.

30. The method of claim 27, wherein the inhibitory construct and the inhibitory signal are administered via a liposome comprising a core and a lipid bilayer, wherein the inhibitory signal is incorporated within the liposome core or lipid bilayer and the inhibitory construct is attached to an outer surface of the liposome.

31. The method of claim 27, wherein the inhibitory construct and inhibitory signal are each chemically linked to a nanocage-based delivery system.

32. The method of claim 27, wherein the inhibitory signal is conjugated to the inhibitory construct via an amine, sulfhydryl or carboxyl functional group present on the inhibitory construct.

33. A vaccine for a mammalian pathogen comprising:
(1) a first composition comprising a peptide comprising at least one copy of a primary immunization composition, said primary immunization composition comprising a recombinant peptide comprising a peptide expressed by the pathogen, wherein said peptide comprises a neutralizing epitope; and
(2) a second composition comprising an inhibitory construct comprising at least one copy of an altered form of the peptide, wherein the altered form comprises at least one amino acid change or structural alteration of the neutralizing epitope that prevents binding of neutralizing antibody thereto; wherein the inhibitory construct is administered prior to, concurrently with or within two months of administration of the primary immunization composition.
